(19) United States
(12) Patent Application Publication
(10) Pub. No.: US 2021/0093346 A1
(43) Pub. Date: Apr. 1, 2021

(54) MEDICAL TREATMENT TOOL

(71) Applicant: OLYMPUS CORPORATION, Tokyo (JP)

(72) Inventor: Masahiro FUJII, Tokyo (JP)

(73) Assignee: OLYMPUS CORPORATION, Tokyo (JP)

(21) Appl. No.: 17/119,549

(22) Filed: Dec. 11, 2020

Related U.S. Application Data
(63) Continuation of application No. PCT/JP2018/023006, filed on Jun. 15, 2018.

(51) Int. Cl. A61B 17/29

(52) U.S. Cl. CPC A61B 17/29; A61B 2017/2924

(57) ABSTRACT

A medical treatment tool includes a tubular sheath having a longitudinal axis, a treatment section supported by a distal end of the sheath in a rotatable manner around the longitudinal axis, an operating section connected to a proximal end of the sheath, and a transmission mechanism that transmits an operational amount applied to the operating section so as to rotate the treatment section around the longitudinal axis. The transmission mechanism includes two transmitting sections that transmit different rotational amounts to the treatment section in accordance with two different kinds of operations performed on the operating section. At least one of the transmitting sections converts an operational amount applied to the operating section along a direction of the longitudinal axis into a rotational amount around the longitudinal axis between the operating section and the treatment section and transmits the rotational amount to the treatment section.

### CROSS-REFERENCE TO RELATED APPLICATIONS

This is a continuation of International Application PCT/JP2018/023006, with an international filing date of Jun. 15, 2018, which is hereby incorporated by reference herein in its entirety.

### TECHNICAL FIELD

The present invention relates to medical treatment tools.

### BACKGROUND ART

A known treatment tool includes a treatment section supported, by the distal end of a flexible tubular sheath having a longitudinal axis, in a rotatable manner around the longitudinal axis (e.g., see Patent Literature 1).

In this treatment tool, the distal end of an operating wire extending through the sheath is connected to the treatment section, having a pair of surgical segments, and the proximal end of the operating wire is connected to an operating section. By operating the operating section to supply a traction force to the operating wire, the pair of surgical segments are opened and closed. By operating the operating section to supply a rotational amount to the operating wire, the treatment section is rotated around the longitudinal axis relative to the sheath.

### CITATION LIST

### Patent Literature

The Publication of Japanese Patent No. 6130134

### SUMMARY OF INVENTION

An aspect of the present invention is directed to a medical treatment tool including a tubular sheath having a longitudinal axis, a treatment section supported by a distal end of the sheath in a rotatable manner around the longitudinal axis, an operating section connected to a proximal end of the sheath, and a transmission mechanism that transmits an operational amount applied to the operating section so as to rotate the treatment section around the longitudinal axis. The transmission mechanism includes two transmitting sections that transmit different rotational amounts to the treatment section in accordance with two different kinds of operations performed on the operating section. At least one of the transmitting sections converts an operational amount applied to the operating section along a direction of the longitudinal axis into a rotational amount around the longitudinal axis between the operating section and the treatment section and transmits the rotational amount to the treatment section.

### BRIEF DESCRIPTION OF DRAWINGS

FIG. 1 is a front view illustrating a medical treatment tool according to an embodiment of the present invention.

FIG. 2 is a vertical sectional view of the medical treatment tool in FIG. 1.

FIG. 3 is an expanded vertical sectional view illustrating the operation of the medical treatment tool in FIG. 1.

FIG. 4 is a vertical sectional view illustrating a first modification of the medical treatment tool in FIG. 1.

FIG. 5 is a side view illustrating an example of a conversion cam used in the medical treatment tool in FIG. 4.

FIG. 6 is a vertical sectional view illustrating a second modification of the medical treatment tool in FIG. 1.

FIG. 7 is a vertical sectional view illustrating a third modification of the medical treatment tool in FIG. 1.

FIG. 8 is a vertical sectional view illustrating a fourth modification of the medical treatment tool in FIG. 1.

FIG. 9 is a vertical sectional view illustrating a fifth modification of the medical treatment tool in FIG. 1.

FIG. 10 is a vertical sectional view illustrating a sixth modification of the medical treatment tool in FIG. 1.

FIG. 11 is a vertical sectional view illustrating a seventh modification of the medical treatment tool in FIG. 1.

FIG. 12 is a vertical sectional view illustrating an eighth modification of the medical treatment tool in FIG. 1.

FIG. 13 is a vertical sectional view illustrating an unlocked state in a ninth modification of the medical treatment tool in FIG. 1.

FIG. 14 is a vertical sectional view illustrating a locked state in the medical treatment tool in FIG. 13.

FIG. 15 is a vertical sectional view illustrating a locked state in a tenth modification of the medical treatment tool in FIG. 1.

FIG. 16 is a vertical sectional view illustrating an unlocked state in the medical treatment tool in FIG. 15.

FIG. 17 is a vertical sectional view illustrating an eleventh modification of the medical treatment tool in FIG. 1.

### DESCRIPTION OF EMBODIMENTS

A medical treatment tool 1 according to an embodiment of the present invention will be described below with reference to the drawings.

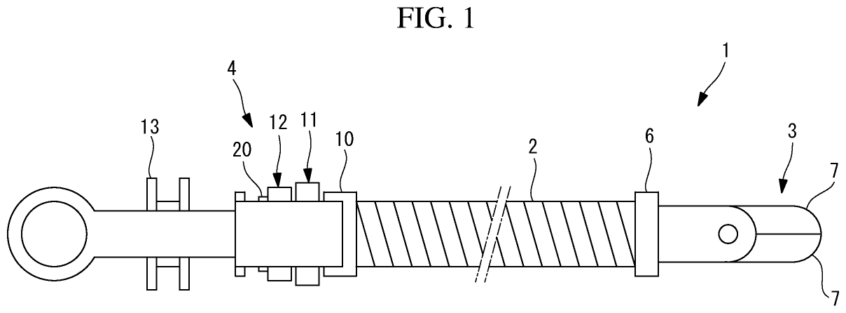

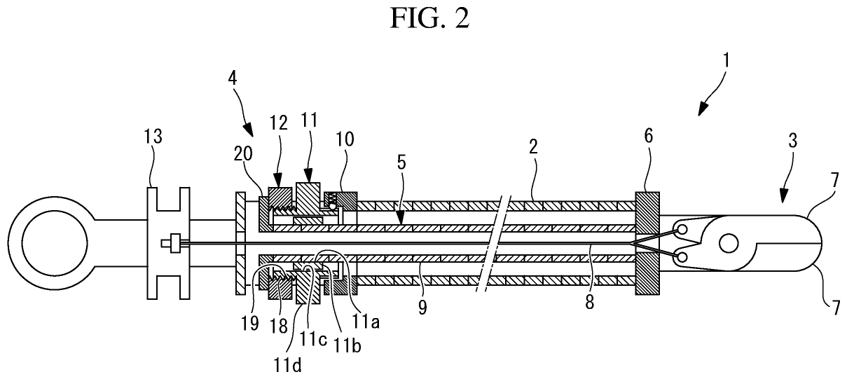

As shown in FIG. 1 and FIG. 2, the medical treatment tool 1 according to this embodiment includes a coil sheath (sheath) 2 having a longitudinal axis, a grasping section (treatment section) 3 supported in a rotatable manner around the longitudinal axis by the distal end of the coil sheath 2, an operating section 4 connected to the proximal end of the coil sheath 2, and a transmission mechanism 5 that transmits a force (operational amount) applied to the operating section 4 so as to rotate the grasping section 3 around the longitudinal axis.

The coil sheath 2 is tubular and has flexibility such that the coil sheath 2 flexibly bends in the bending direction.

The grasping section 3 includes a rotating base 6 disposed in a slidable manner around the longitudinal axis relative to a distal-end surface of the coil sheath 2, and also includes two grasping segments 7 supported by the rotating base 6 in a swivelable manner around an axis orthogonal to the longitudinal axis. The proximal ends of the grasping segments 7 are connected to a wire 8 that transmits a traction force applied to the proximal end of the coil sheath 2. The two grasping segments 7 are swiveled by the traction force from the wire 8, so that a surgical target can be grasped between the grasping segments 7.

In this embodiment, the transmission mechanism 5 is constituted of a twisted wire (transmitting section) 9 having torque transmissibility. The twisted wire 9 extends through the coil sheath 2 in the longitudinal direction, has a distal end thereof fixed to the rotating base 6, and has a proximal end thereof connected to the operating section 4.

When the twisted wire 9 receives a rotational torque acting around the longitudinal axis from the operating section 4 connected to the proximal end of the twisted wire 9, the twisted wire 9 transmits the rotational torque to the distal end thereof to rotate the rotating base 6 around the longitudinal axis.

On the other hand, when the twisted wire 9 receives a traction force acting along the longitudinal axis from the operating section 4 connected to the proximal end, the twisted wire 9 becomes untwisted by the traction force, thus causing the distal end thereof to be rotated in the untwisting direction. Specifically, the twisted wire 9 functions as two transmitting sections that transmit different rotational amounts (rotational angles) to the grasping section 3 in accordance with two kinds of operational amounts applied to the proximal end by the operating section 4.

The operating section 4 includes an operating body 10 fixed to the proximal end of the coil sheath 2, a first dial (second input section) 11 supported in a rotatable manner around the longitudinal axis relative to the operating body 10, and a second dial (first input section) 12. A slider 13 is attached to the operating body 10 in a movable manner along the longitudinal axis. The slider 13 is connected to the proximal end of the wire 8 connected to the grasping section 3.

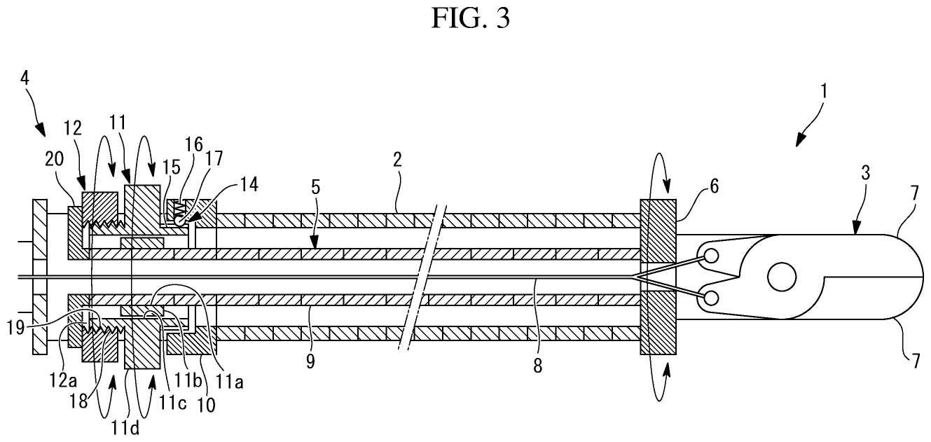

As shown in FIG. 3, the first dial 11 and the second dial 12 are ring-shaped with center holes 11a and 12a, respectively. The twisted wire 9 extends through the center holes 11a and 12a.

The first dial 11 includes a cross-sectionally square-shaped inner member 11b fixed to the outer peripheral surface of the twisted wire 9 extending through the center hole 11a, and also includes an outer member 11d having a fitting hole 11c with a size slightly larger than the outer diameter of the inner member 11b. It is preferable that there is a slight gap between the outer surface of the inner member 11b and the inner surface of the outer member 11d when the inner member 11b is fitted to the fitting hole 11c of the outer member 11d. Accordingly, the first dial 11 moves along the longitudinal axis without interfering with the twisted wire 9 and moves around the longitudinal axis in synchronization therewith.

Furthermore, the first dial 11 is provided with a ratchet mechanism 14 for preventing the first dial 11 from rotating around the longitudinal axis when the first dial 11 is not receiving a predetermined rotational amount or more. For example, as shown in FIG. 3, the ratchet mechanism 14 includes a plurality of recesses 15 arranged in the circumferential direction in the outer peripheral surface of the first dial 11, and also includes a ball 17 that is biased radially inward by a spring 16 from the radially outer side of the recesses 15 and that engages with any of the recesses 15. When the ball 17 is engaged with the recess 15, the first dial 11 is prevented from rotating around the longitudinal axis. When the first dial 11 receives a predetermined rotational amount or more in this state, the ball 17 moves away from the recess 15 so that the locked state becomes released, whereby the first dial 11 can be rotated around the longitudinal axis.

The proximal end of the first dial 11 is provided with a male thread 18 extending along the longitudinal axis, and the second dial 12 is provided with a female thread 19 engaged with the male thread 18 of the first dial 11. Accordingly, when the second dial 12 is rotated around the longitudinal axis in a state where the first dial 11 is stationary, the second dial 12 moves along the longitudinal axis while rotating around the longitudinal axis relative to the first dial 11.

A flanged engagement segment 20 extending radially outward is fixed to the proximal end of the twisted wire 9. The engagement segment 20 is in contact with the proximal end surface of the second dial 12 in a slidable manner. Accordingly, when the second dial 12 is rotated around the longitudinal axis relative to the first dial 11, the second dial 12 moves along the longitudinal axis toward, for example, the proximal end. As a result, the engagement segment 20 in contact with the proximal end surface of the second dial 12 is pressed toward the proximal end, so that a traction force acting toward the proximal end is input to the proximal end of the twisted wire 9 fixed to the engagement segment 20.

Specifically, when the first dial 11 is rotated around the longitudinal axis relative to the operating body 10, the rotational amount is directly input to the twisted wire 9 fixed to the first dial 11, so that the rotational amount around the longitudinal axis is input to the proximal end of the twisted wire 9. On the other hand, when the second dial 12 is rotated around the longitudinal axis relative to the first dial 11, the engagement between the male thread 18 and the female thread 19 causes the rotational amount to be converted into an operational amount that causes the second dial 12 to move along the longitudinal axis. As a result, a traction force acting along the longitudinal axis is input to the twisted wire 9 via the engagement segment 20 pressed by the second dial 12.

In a case where the grasping section 3 is rotated around the longitudinal axis utilizing the twist of the twisted wire 9 by inputting a traction force to the proximal end of the twisted wire 9, a small rotational amount acts on the grasping section 3, as compared with a case where the grasping section 3 fixed to the distal end of the twisted wire 9 is rotated around the longitudinal axis by inputting a rotational amount to the proximal end of the twisted wire 9. Therefore, when the first dial 11 is rotated around the longitudinal axis relative to the operating body 10, the grasping section 3 undergoes relatively large coarse movement. In contrast, when the second dial 12 is rotated around the longitudinal axis relative to the first dial 11, the grasping section 3 undergoes relatively small fine movement.

Specifically, the transmission mechanism 5 includes two transmitting sections that transmit different rotational amounts to the grasping section 3 in accordance with two different kinds of operations performed on the operating section 4.

Accordingly, the medical treatment tool 1 according to this embodiment uses a combination of rough positioning of the grasping section 3 around the longitudinal axis by operating the first dial 11 and fine positioning of the grasping section 3 around the longitudinal axis by operating the second dial 12, so that the grasping section 3 disposed at the distal end of the flexible coil sheath 2 can be rotated around the longitudinal axis of the coil sheath 2.

Tension is transmitted more reliably by the twisted wire 9 even when the flexible coil sheath 2 is bent, so that fine movement of the grasping section 3 can be performed more reliably. Specifically, this is advantageous in that, by combining the relatively large coarse movement in which the rotational amount is directly transmitted and the relatively small fine movement in which the transmitted tension (operational amount) is converted into a rotational amount, the grasping section 3 can be rotated smoothly, regardless of the bending of the coil sheath 2.

As an alternative to this embodiment in which the inner member 11b used is a cross-sectionally square-shaped member as a structure where the first dial 11 moves along the longitudinal axis without interfering with the twisted wire 9 and moves around the longitudinal axis in synchronization therewith, a cross-sectionally polygonal member, a cross-sectionally circular member having a cutout, or a cross-sectionally circular member having a protrusion may be used.

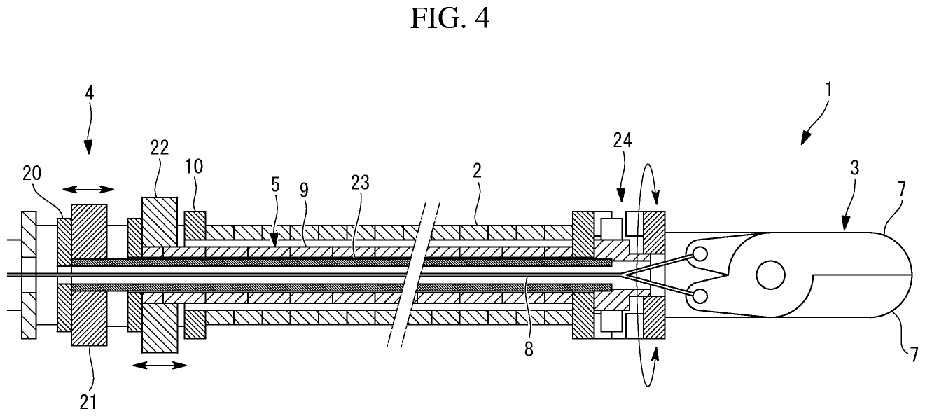

Furthermore, as an alternative to this embodiment in which different rotational amounts are transmitted to the grasping section 3 in accordance with two different kinds of operations involving rotating the two dials 11 and 12 provided in the operating section 4 around the longitudinal axis, the grasping section 3 may be rotated around the longitudinal axis by moving two handles 21 and 22 along the longitudinal axis, as shown in FIG. 4.

Specifically, in the example shown in FIG. 4, the transmission mechanism 5 includes the twisted wire 9, a flexible tube (transmitting section) 23 that extends through the twisted wire 9 along the longitudinal axis and that transmits an operational amount in the direction of the longitudinal axis, and a conversion cam 24 that is disposed between the distal end of the tube 23 and the grasping section 3 and that converts the operational amount in the direction of the longitudinal axis and transmitted by the tube 23 into a rotational amount. The first handle (first input section) 21 is fixed to the proximal end of the tube 23. By moving the first handle 21 along the longitudinal axis, an operational amount in the direction of the longitudinal axis is supplied to the tube 23, so that the grasping section 3 can be rotated (coarsely moved) around the longitudinal axis by a relatively large rotational angle.

On the other hand, the second handle (second input section) 22 is fixed to the proximal end of the twisted wire 9. By moving the second handle 22 along the longitudinal axis, a traction force can be applied to the twisted wire 9. With a rotational amount occurring in a direction that causes the twisted wire 9 to become untwisted due to the traction force transmitted through the twisted wire 9, the grasping section 3 can be rotated (finely moved) around the longitudinal axis by a relatively small rotational angle.

Specifically, the conversion efficiency varies between the conversion cam 24 that converts an operational amount applied to the tube 23 along the longitudinal axis into a rotational amount and the twisted wire 9 that converts a traction force applied to the proximal end thereof into a rotational amount at the distal end thereof, so that relatively large coarse movement can be realized by the conversion cam 24 and relatively small fine movement can be realized by the twisted wire 9.

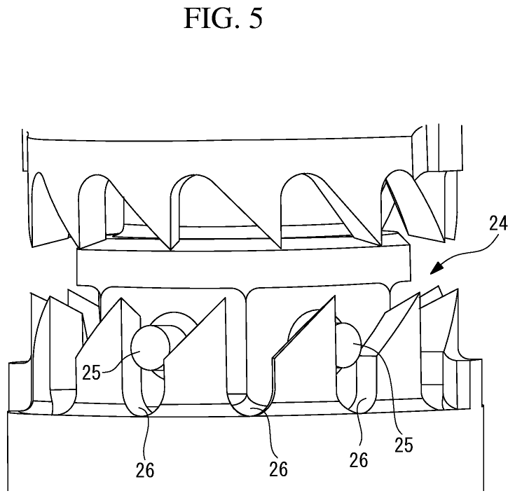

For example, as shown in FIG. 5, the conversion cam 24 includes protrusions 25 protruding radially from the distal end of the tube 23, and also includes a plurality of cam recesses 26 disposed apart from each other in the circumferential direction at the proximal end of the grasping section 3 and capable of accommodating the protrusions 25 therein. When each protrusion 25 makes one reciprocation along the longitudinal axis, the corresponding cam recess 26 accommodating the protrusion 25 is switched to the neighboring cam recess 26 in the circumferential direction, so that the grasping section 3 can be intermittently rotated around the longitudinal axis.

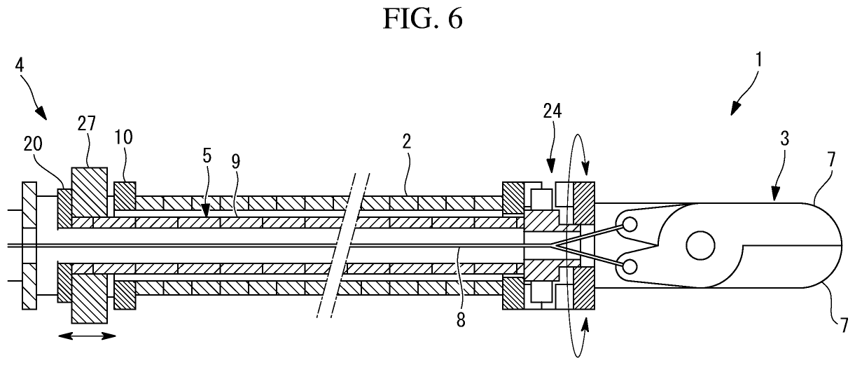

Furthermore, as an alternative to the above-described case where the grasping section 3 is coarsely moved in accordance with an operational amount transmitted by the tube 23 along the longitudinal axis and the grasping section 3 is finely moved by a traction force transmitted by the twisted wire 9, the grasping section 3 may be coarsely moved by the conversion cam 24 in accordance with an operational amount applied to the twisted wire 9 along the longitudinal axis by a single handle (input section) 27 fixed to the proximal end of the twisted wire 9, and the grasping section 3 may be finely moved in accordance with a traction force applied to the twisted wire 9 by the handle 27, as shown in FIG. 6.

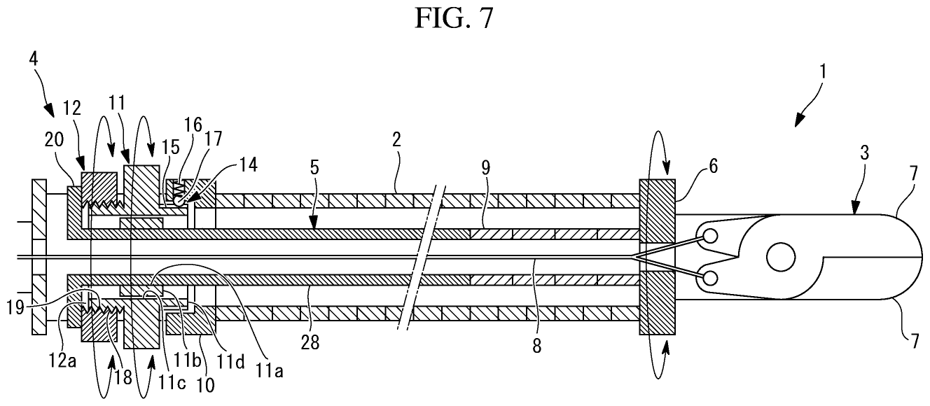

Furthermore, as an alternative to the twisted wire 9 that receives a rotational amount and a traction force from the two dials 11 and 12, a tube (transmitting section) 28 capable of transmitting a rotational amount and a traction force to an intermediate position in the direction of the longitudinal axis may be used, and the twisted wire 9 may be disposed between the tube 28 and the grasping section 3, as shown in FIG. 7. Reducing the length of the twisted wire 9 makes it easier to transmit the rotational amount and can limit the rotational amount for the fine movement by the twisted wire 9.

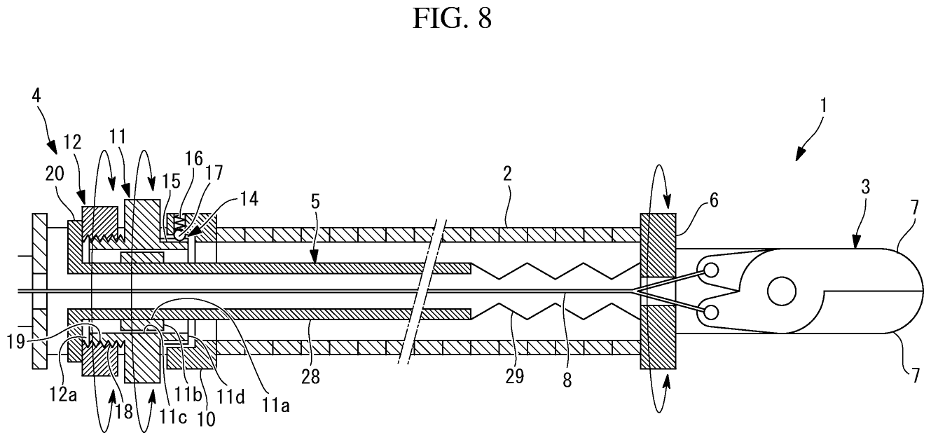

In this case, as shown in FIG. 8, the twisted wire 9 may be replaced with a tubular member (transmitting section) 29 formed by folding paper into a shape that causes a rotational amount to be generated at the distal end by being stretched by a traction force.

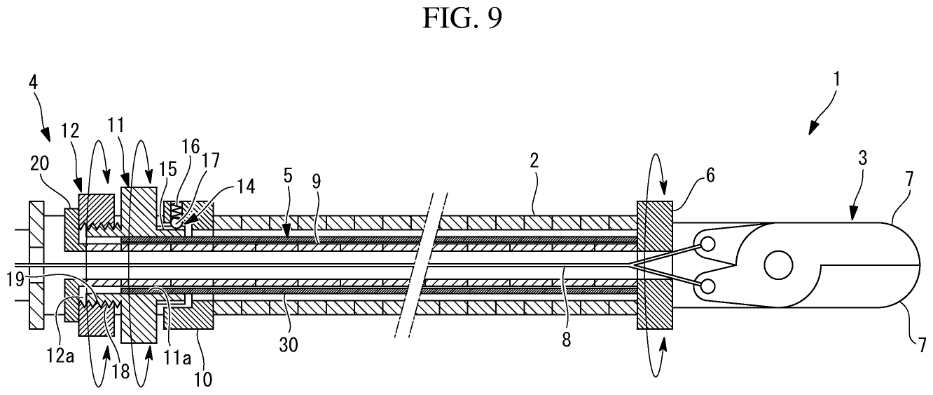

Furthermore, as shown in FIG. 9, the rotational amount may be transmitted by a tube (transmitting section) 30, and the traction force may be transmitted by the twisted wire 9. Specifically, the tube 30, which is capable of transmitting torque over the entire length of the coil sheath 2, may be disposed between the first dial 11 and the grasping section 3. This is advantageous in that the compressive resistance along the longitudinal axis can be improved, as compared with the case where the twisted wire 9 alone is provided.

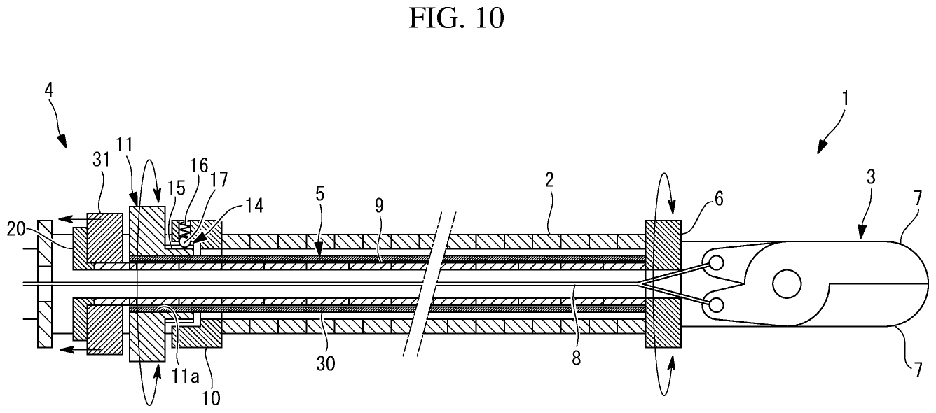

In this case, as shown in FIG. 10, a rotational amount may be input by rotating the first dial 11 fixed to the proximal end of the tube 30 around the longitudinal axis, and a traction force may be input by moving a handle (first input section) 31 fixed to the proximal end of the twisted wire 9 along the longitudinal axis. The coarse movement and the fine movement can be realized by different operations, so that erroneous operations can be prevented.

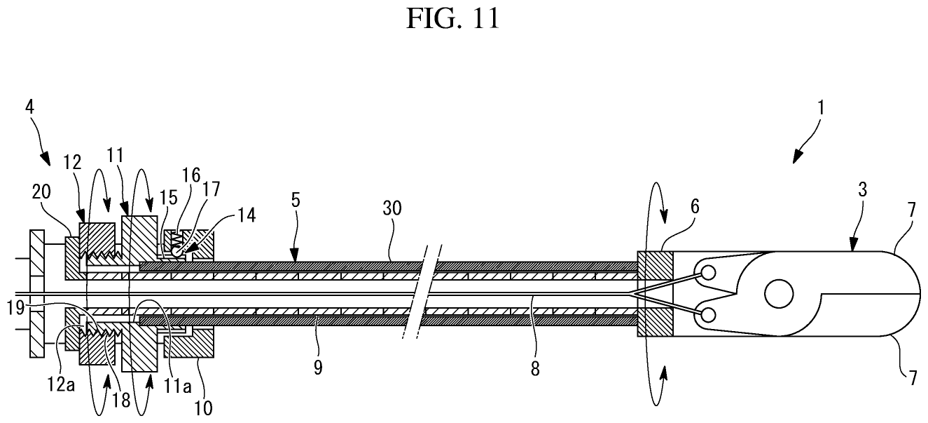

As an alternative to FIG. 9 in which the tube 30 is disposed within the coil sheath 2, the coil sheath 2 may be omitted, and a sheath may be constituted of the tube 30 (other transmitting section) alone, as shown in FIG. 11.

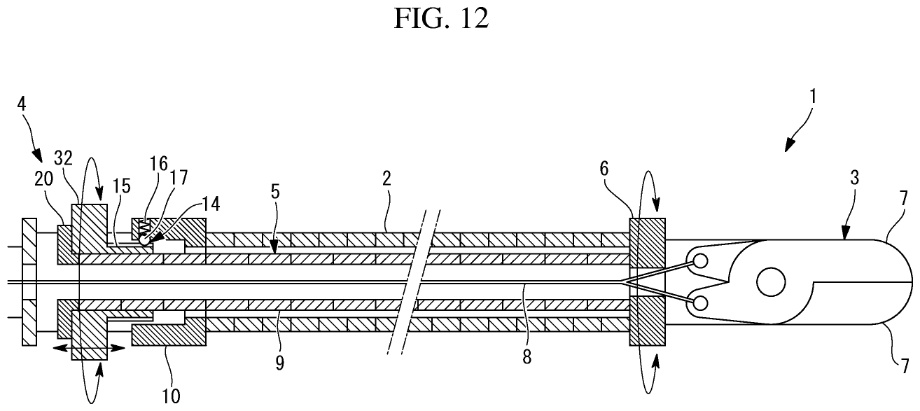

Furthermore, as an alternative to FIG. 3 in which the first dial 11 that inputs a rotational amount to the twisted wire 9 and the second dial 12 that inputs a traction force to the twisted wire 9 are separately provided, a rotational amount and a traction force may be inputtable using a single dial (input section) 32, as shown in FIG. 12. In this case, since the dial 32 moves along the longitudinal axis, the recesses 15 constituting the ratchet mechanism 14 also need to be long in the direction of the longitudinal axis.

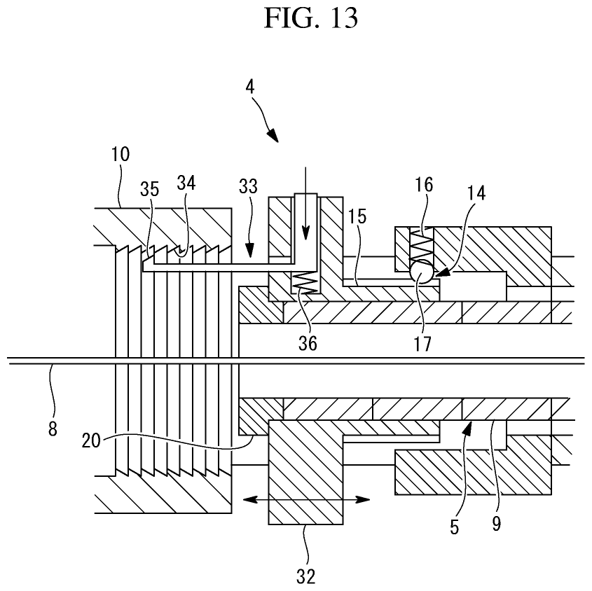

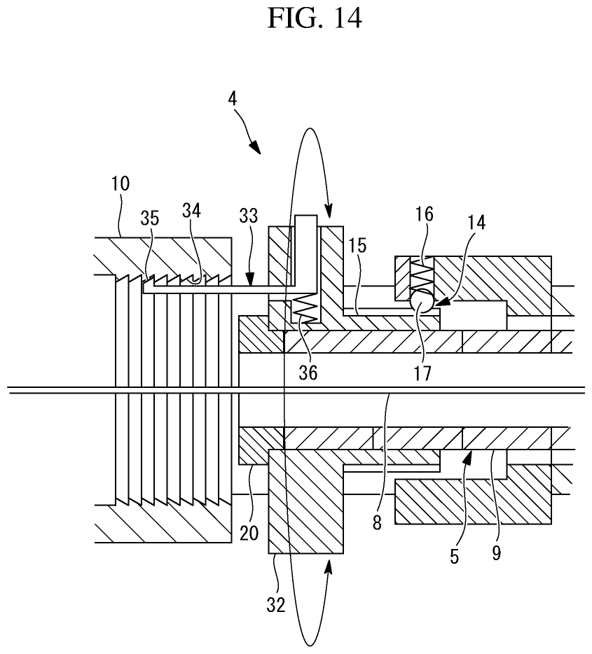

Furthermore, in this case, it is preferable that a lock mechanism 33 capable of locking the dial 32 at each position in the direction of the longitudinal axis be provided, as shown in FIG. 13 and FIG. 14. In the example shown in FIG. 13 and FIG. 14, a plurality of peripheral grooves 34 are arranged along the longitudinal axis in the operating body 10, and the dial 32 is provided with an engagement segment 35 that engages with any of the peripheral grooves 34. Reference sign 36 denotes a spring that biases the engagement segment 35 in a direction in which the engagement segment 35 engages with the peripheral groove 34.

As shown in FIG. 13, the engagement segment 35 and the peripheral groove 34 are disengaged from each other by pressing the engagement segment 35 against the spring 36, so that the dial 32 can be moved along the longitudinal axis. As shown in FIG. 14, the dial 32 can be stopped at each position in the longitudinal direction by engaging the engagement segment 35 with any of the peripheral grooves 34 at a freely-chosen position.

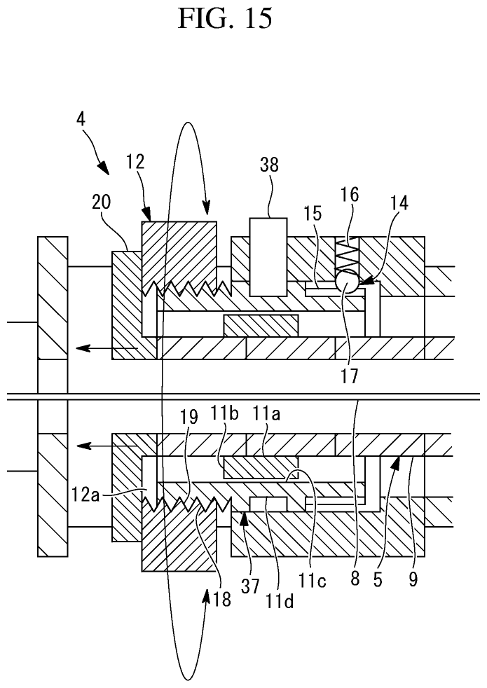

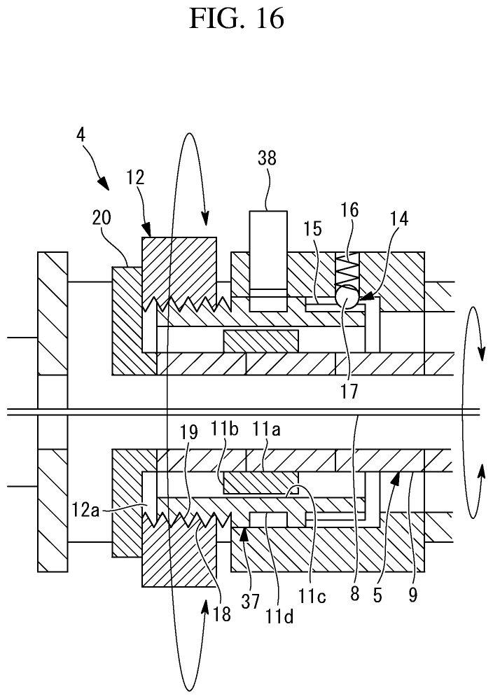

Furthermore, as shown in FIG. 15 and FIG. 16, in place of the first dial 11, a tubular member 37 fixed to the proximal end of the twisted wire 9 may be provided with the male thread 18 engaged with the female thread 19 of the second dial (input section) 12, and a lock mechanism (switching section) 38 capable of fixing the tubular member 37 at a freely-chosen position may be provided.

In this case, as shown in FIG. 16, when the lock mechanism 38 is in an unlocked state, the tubular member 37 is rotatable around the longitudinal axis, and is rotated around the longitudinal axis in response to an operation performed on the second dial 12 due to friction between the male thread 18 and the female thread 19 that are engaged with each other. On the other hand, as shown in FIG. 15, when the lock mechanism 38 is in an active state, the tubular member 37 is prevented from rotating around the longitudinal axis. When the second dial 12 is rotated around the longitudinal axis, the male thread 18 of the tubular member 37 and the female thread 19 of the second dial 12 rotate relatively to each other, so that the second dial 12 moves along the longitudinal axis, whereby a traction force can be input to the twisted wire 9. Accordingly, it is possible to switch between an input of a rotational amount and an input of a traction force by operating a single second dial 12.

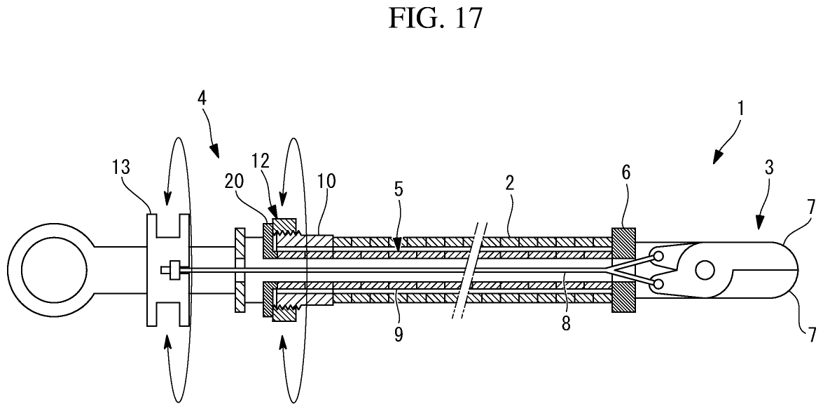

Furthermore, in this embodiment, as shown in FIG. 17, the grasping section 3 may be coarsely moved by rotating the wire 8, which opens and closes the grasping section 3, around the longitudinal axis.

Furthermore, as an alternative to the above embodiment in which the grasping section 3 is provided at the distal end, the above embodiment may be applied to a medical treatment tool 1 equipped with another freely-chosen treatment section.

As a result, the above-described embodiment leads to the following aspect.

An aspect of the present invention is directed to a medical treatment tool including a tubular sheath having a longitudinal axis, a treatment section supported by a distal end of the sheath in a rotatable manner around the longitudinal axis, an operating section connected to a proximal end of the sheath, and a transmission mechanism that transmits an operational amount applied to the operating section so as to rotate the treatment section around the longitudinal axis. The transmission mechanism includes two transmitting sections that transmit different rotational amounts to the treatment section in accordance with two different kinds of operations performed on the operating section. At least one of the transmitting sections converts an operational amount applied to the operating section along a direction of the longitudinal axis into a rotational amount around the longitudinal axis between the operating section and the treatment section and transmits the rotational amount to the treatment section.

According to this aspect, when the operating section is operated at the proximal end of the sheath, the operational amount applied to the operating section is transmitted by the transmission mechanism, so that the treatment section is rotated around the longitudinal axis at the distal end of the sheath. In this case, different rotational amounts are transmitted to the treatment section via the two transmitting sections in accordance with the two different kinds of operations performed on the operating section. Accordingly, by switching the operation performed on the operating section, it is possible to switch between coarse movement in which the treatment section is rotated by a large amount by receiving a large rotational amount and fine movement in which the treatment section is rotated by receiving a small rotational amount.

Furthermore, since at least one of the transmitting sections converts an operational amount in the direction of the longitudinal axis into a rotational amount around the longitudinal axis while transmitting the operational amount, a force transmitted effectively to near the treatment section by means of the operational amount in the direction of the longitudinal axis is converted into a rotational amount even in a condition where a rotational amount is less likely to be transmitted due to bending of the sheath, whereby the treatment section disposed at the distal end of the flexible sheath can be rotated smoothly around the longitudinal axis of the sheath.

In the above aspect, an other transmitting section may transmit a rotational amount applied to the operating section directly to the treatment section.

According to this configuration, coarse movement in which the treatment section is rotated by a large amount around the longitudinal axis can be performed in accordance with the rotational amount applied to the operating section, and fine movement in which the treatment section is rotated by a small amount around the longitudinal axis can be performed in accordance with the operational amount applied to the operating section along the longitudinal axis.

Furthermore, in the above aspect, the operating section may include an operating body fixed to the proximal end of the sheath, a first input section supported in a movable manner along the direction of the longitudinal axis relative to the operating body, and a second input section supported in a rotatable manner around the longitudinal axis relative to the operating body. The first input section may be connected to the at least one transmitting section, and the second input section may be connected to the other transmitting section.

According to this configuration, the first input section is moved along the longitudinal axis relative to the operating body. Thus, the operational amount in the direction of the longitudinal axis is transmitted via one of the transmitting sections and is converted into a rotational amount around the longitudinal axis while being transmitted, so that the treatment section can be rotated around the longitudinal axis. Furthermore, by rotating the second input section around the longitudinal axis relative to the operating body, the rotational amount is transmitted directly to the treatment section via the other transmitting section, so that the treatment section can be rotated around the longitudinal axis.

Furthermore, in the above aspect, the two transmitting sections may convert two kinds of operational amounts applied to the operating section along the direction of the longitudinal axis into rotational amounts around the longitudinal axis at different conversion efficiencies between the operating section and the treatment section and may transmit the rotational amounts to the treatment section.

According to this configuration, the two kinds of operational amounts applied to the operating section along the longitudinal axis are converted into rotational amounts around the longitudinal axis at different conversion efficiencies by the two transmitting sections. The treatment section is coarsely moved by the transmitting section with the higher conversion efficiency, whereas the treatment section is finely moved by the transmitting section with the lower conversion efficiency.

Furthermore, in the above aspect, the operating section may include an operating body fixed to the proximal end of the sheath and an input section supported in a movable manner along the direction of the longitudinal axis relative to the operating body. The transmission mechanism may vary the conversion efficiency in accordance with a position of the input section along the direction of the longitudinal axis.

According to this configuration, different rotational amounts can be transmitted to the treatment section by the two transmitting sections having different conversion efficiencies in accordance with the position of the single input section along the longitudinal axis.

Furthermore, in the above aspect, the operating section may convert a rotational amount around the longitudinal axis into an operational amount along the direction of the longitudinal axis.

According to this configuration, when a rotational amount around the longitudinal axis is applied to the operating section, the rotational amount is converted into an operational amount in the direction of the longitudinal axis by the operating section, and the operational amount is transmitted to the treatment section by the transmission mechanism.

Furthermore, in the above aspect, the operating section may include an operating body fixed to the proximal end of the sheath and a single input section supported in a rotatable manner around the longitudinal axis relative to the operating body. The medical treatment tool may further include a switching section that selectively switches an input of a rotational amount to the input section to an input to either one of the two transmitting sections.

According to this configuration, of the two transmitting sections, only one of the transmitting sections switched by the switching section can receive the rotational amount applied to the input section, so that coarse movement and fine movement can reliably be performed independently of each other.

Furthermore, in the above aspect, the other transmitting section may include the sheath.

According to this configuration, the sheath can function as the other transmitting section, thereby achieving a reduced number of components, a simplified structure, and a reduced diameter.

Furthermore, in the above aspect, the switching section may include a lock mechanism that selectively prevents two input sections from rotating relative to the operating body.

According to this configuration, the lock mechanism is activated so that either one of the input sections is selectively prevented from rotating. By inputting a rotational amount to the non-locked input section, coarse movement or fine movement can be independently performed.

The present invention is advantageous in that it can rotate a treatment section disposed at the distal end of a flexible sheath smoothly around a longitudinal axis of the sheath.

### REFERENCE SIGNS LIST

1 medical treatment tool
2 coil sheath (sheath)
3 grasping section (treatment section)
4 operating section
5 transmission mechanism
9 twisted wire (transmitting section)
10 operating body
11 first dial (second input section)
12 second dial (first input section, input section)
21 first handle (first input section)
22 second handle (second input section)
23, 28, 30 tube (transmitting section, sheath)
27, 31 handle (input section, first input section)
29 tubular member (transmitting section)
32 dial (input section)
38 lock mechanism (switching section)

1. A medical treatment tool comprising:
a tubular sheath having a longitudinal axis;
a treatment section supported by a distal end of the sheath in a rotatable manner around the longitudinal axis;
an operating section connected to a proximal end of the sheath; and
a transmission mechanism that transmits an operational amount applied to the operating section so as to rotate the treatment section around the longitudinal axis,
wherein the transmission mechanism includes two transmitting sections that transmit different rotational amounts to the treatment section in accordance with two different kinds of operations performed on the operating section, and
wherein at least one of the transmitting sections converts an operational amount applied to the operating section along a direction of the longitudinal axis into a rotational amount around the longitudinal axis between the operating section and the treatment section and transmits the rotational amount to the treatment section.

2. The medical treatment tool according to claim 1,
wherein an other transmitting section different from the one transmitting section transmits a rotational amount applied to the operating section directly to the treatment section.

3. The medical treatment tool according to claim 2,
wherein the operating section comprises an operating body fixed to the proximal end of the sheath, a first input section supported in a movable manner along the direction of the longitudinal axis relative to the operating body, and a second input section supported in a rotatable manner around the longitudinal axis relative to the operating body,
wherein the first input section is connected to the one transmitting section, and
wherein the second input section is connected to the other transmitting section.

4. The medical treatment tool according to claim 1,
wherein the two transmitting sections convert two kinds of operational amounts applied to the operating section along the direction of the longitudinal axis into rotational amounts around the longitudinal axis at different conversion efficiencies between the operating section and the treatment section and transmit the rotational amounts to the treatment section.

5. The medical treatment tool according to claim 4,
wherein the operating section comprises an operating body fixed to the proximal end of the sheath, and an input section supported in a movable manner along the direction of the longitudinal axis relative to the operating body, and
wherein the transmission mechanism varies the conversion efficiency in accordance with a position of the input section along the direction of the longitudinal axis.

6. The medical treatment tool according to claim 1,
wherein the operating section converts a rotational amount around the longitudinal axis into an operational amount along the direction of the longitudinal axis.

7. The medical treatment tool according to claim 6,
wherein the operating section comprises an operating body fixed to the proximal end of the sheath, and two input sections supported independently in a rotatable manner around the longitudinal axis relative to the operating body.

8. The medical treatment tool according to claim 6,
wherein the operating section comprises an operating body fixed to the proximal end of the sheath, and a single input section supported in a rotatable manner around the longitudinal axis relative to the operating body, and
wherein the medical treatment tool further comprises a switching section that selectively switches an input of a rotational amount to the input section to an input to either one of the two transmitting sections.

9. The medical treatment tool according to claim 1,
wherein an other transmitting section comprises the sheath.

10. The medical treatment tool according to claim 8,
wherein the switching section comprises a lock mechanism that selectively prevents two input sections from rotating relative to the operating body.

11. The medical treatment tool according to claim 1,
wherein the transmitting sections comprise a twisted wire, and
wherein the transmitting sections convert an operational amount applied to the operating section along the direction of the longitudinal axis into a rotational amount around the longitudinal axis between the operating section and the treatment section and transmit the rotational amount to the treatment section, and
wherein the transmitting sections transmit an operational amount applied to the operating section around the longitudinal axis as a rotational amount larger than the rotational amount around the longitudinal axis.
